(19) United States
(12) Patent Application Publication
(10) Pub. No.: US 2026/0215665 A1
(43) Pub. Date: Jul. 30, 2026

(54) SYSTEM AND METHOD FOR AUTOMATIC STOMACH SCREENING

(71) Applicant: MULTI-SCALE MEDICAL ROBOTICS CENTER LIMITED, Hong Kong (CN)

(72) Inventors: Zheng LI, Hong Kong (CN); Yehui LI, Hong Kong (CN); Yichong SUN, Hong Kong (CN); Wai Yan Philip CHIU, Hong Kong (CN); Yun Wong James LAU, Hong Kong (CN)

(21) Appl. No.: 19/157,356

(22) PCT Filed: Feb. 16, 2024
(86) PCT No.: PCT/IB2024/051471

§ 371 Date: Aug. 16, 2025

Related U.S. Application Data
(60) Provisional application No. 63/446,024, filed on Feb. 16, 2023.

(51) Int. Cl. A61B 1/04; A61B 1/00

(52) U.S. Cl. CPC A61B 1/041; A61B 1/000096; A61B 1/00158

(57) ABSTRACT

This invention provides a system for imaging one or more specific structures in a stomach within a workspace. In one embodiment, said system comprises: a) an active capsule endoscope to be placed into said stomach, comprising a camera, an inertial measurement unit and a permanent magnet; b) an actuation unit comprising a Hall-effect sensor array located at bottom of said workspace and a plurality of magnetic generators for generating a desired magnetic field; and c) a controller unit.

### FIELD OF THE INVENTION

The invention relates to a system and a method for automatic stomach screening, particularly, a system involving an active capsule endoscope and a magnetic actuation unit to conduct active screening inside the stomach, and a method controlling the active capsule endoscope to conduct automatic stomach screening by subsequently viewing six major parts of the stomach with the assistance of an image artificial intelligence model and closed-loop motion controllers.

### BACKGROUND OF THE INVENTION

Stomach cancer is one of the most severe health challenges with a high incidence and mortality, which has more than one million cases and 0.76 million deaths reported in 2020 all over the world. To date, regular screening and early diagnosis represents the most significant way to prevent stomach cancer. Nevertheless, mass screening programs for stomach cancer is still limited due to the shortage of endoscopists, particularly in developing countries. Wireless capsule endoscopy provides a non-invasive diagnosis methodology for the gastrointestinal (GI) tract, especially in stomach.

Currently, Wireless capsule endoscopy can be divided into two major types based on its motion properties: (1) passive capsule endoscopes (PCEs) and (2) active capsule endoscopes (ACEs). There are a lot of PCEs that have been developed by researchers and commercial companies, such as in US Patent NO. U.S. Pat. No. 7,244,229B2, entitled “Capsule endoscope” filed on Aug. 10, 2005, and in U.S. Pat. No. 7,316,647B2, entitled “Capsule endoscope and a capsule endoscope system” filed on Apr. 23, 2004. The PCEs move passively relying on peristaltic wave of GI tract and transmit captured images to surgeons for further diagnosis. However, such passive movement limits availability of region-of-interest (ROI) diagnosis, which thus lowers the accuracy and reliability of diagnosis. Whereas ACEs can achieve active locomotion inside the GI tract with various locomotive mechanisms, such as legged propulsion (U.S. Pat. No. 8,066,632B2 entitled “Teleoperated endoscopic capsule equipped with active locomotion system” filed on Feb. 17, 2005), inchworm-like advancement (U.S. Pat. No. 7,365,509B2 entitled “Capsule type micro-robot moving system” filed on May 10, 2006), and magnetic actuation (U.S. Pat. No. 9,125,557B2 entitled “Magnetic maneuvering system for capsule endoscope” filed on May 30, 2012). Among these locomotive approaches, magnetic actuation shows promising potentials in clinical applications, which transmits functional torques and forces to the capsule endoscope and thus control its motion through magnetic fields instead of other bulky on-board mechanisms. Due to the advantages of magnetic actuation, there are also some magnetically actuated capsule endoscopes (MACEs) with with various functionalities, such as biopsy (U.S. Pat. No. 9,125,557B2 entitled “Magnetically actuated capsule endoscope, magnetic field generating and sensing apparatus and method of actuating a magnetically actuated capsule endoscope” filed on May 23, 2018) and drug delivery (U.S. Pat. No. 9,445,711B2 entitled “System and method to magnetically actuate a capsule endoscopic robot for diagnosis and treatment” filed on May 9, 2013), which enhance their applicability in clinical practice.

Typically, higher maneuverability of MACEs can lead to more comprehensive diagnosis and shorter procedure duration, thus energy from the embedded battery can be consumed more on real-time images transmission with a high resolution instead of ensuring long endurance with low-quality and low-frame-rate camera view. The maneuverability of developed MACEs varies depending on their actuation systems, which can be divided into two major types: (1) permanent-magnet-based actuation (PMBA) systems; (2) electromagnetic actuation (EMA) systems. For PMBA systems, a robotic manipulator is typically utilized to carry the permanent magnet to generate desired magnetic fields, which thus realizes multiple degree-of-freedom (DOF) motion control of MACEs. For instance, Mohoney et al. (Mahoney et al., “Five-degree-of-freedom manipulation of an untethered magnetic device in fluid using a single permanent magnet with application in stomach capsule endoscopy.” The International Journal of Robotics Research 35.1-3 (2016): 129-147.) utilized a single permanent magnet mounted on a robot arm to achieve 5-DOF closed-loop control (i.e., 3-DOF position and 2-DOF orientation control) of a capsule robot in a liquid environment with a high accuracy. However, such a method has controllability limitations caused by one-directional attraction forces from the large external permanent magnet, which thus leads to insufficient maneuverability, unsatisfactory motion response time, and complicated maneuvering processes.

Compared to PMBA systems, EMA systems can more easily fulfill multi-DOF motion control of MACEs by changing the currents applied to multiple electromagnets. Hoang et al. (Hoang et al. “Independent electromagnetic field control for practical approach to actively locomotive wireless capsule endoscope.” IEEE Transactions on Systems, Man, and Cybernetics: Systems 51.5 (2019): 3040-3052.) and Lee et al. (Lee et al. “Active locomotive intestinal capsule endoscope (ALICE) system: A prospective feasibility study.” IEEE/ASME Transactions on Mechatronics 20.5 (2014): 2067-2074.) realized 5-DOF open-loop motion control of MACEs by combining a uniform magnetic field and a gradient magnetic field generated by multiple pairs of hollow electromagnetic coils. With further development, Song et al. (Song et al. “Motion Control of Capsule Robot Based on Adaptive Magnetic Levitation Using Electromagnetic Coil.” IEEE Transactions on Automation Science and Engineering (2022).) achieved simultaneous levitation and orientation closed-loop control of a capsule robot. The authors used three pairs of hollow Helmholtz coils to generate uniform magnetic fields and a 3-DOF movable electromagnetic coil to generate gradient magnetic fields, which thus achieved more dexterous motion control of MACEs. Compared to hollow electromagnetic coils used by aforementioned EMA systems, combination of iron cores and coils can generate stronger magnetic fields than that of air-core-based electromagnets, which could result in less energy consumption and heat generation. For example, Kummer et al. (Kummer et al. “OctoMag: An electromagnetic system for 5-DOF wireless micromanipulation.” IEEE Transactions on Robotics 26.6 (2010): 1006-1017.) reported a well-designed iron-core-based EMA system that can generate non-uniform magnetic fields and fulfill 5-DOF motion control of a magnetic object. Nevertheless, most EMA systems either have limited-DOF pose feedback of MACEs or use external cameras to recognize the capsule's pose that is not realistic for closed-loop control in clinical environment. A feasible and accurate in-vivo localization solution should be integrated in these systems to fulfill multi-DOF closed-loop control of MACEs. Some related works on magnetic localization are listed as follows:
a. Son et al. “A 5-D localization method for a magnetically manipulated untethered robot using a 2-D array of Hall-effect sensors.” IEEE/ASME transactions on mechatronics 21.2 (2015): 708-716.
b. Dai et al. “A novel 6-D tracking method by fusion of 5-D magnetic tracking and 3-D inertial sensing.” IEEE Sensors Journal 18.23 (2018): 9640-9648.
c. Zhang et al. “6-D spatial localization of wireless magnetically actuated capsule endoscopes based on the fusion of hall sensor array and IMU.” IEEE Sensors Journal 22.13 (2022): 13424-13433.
d. Placidi et al. “Review on patents about magnetic localisation systems for in vivo catheterizations.” Recent Patents on Biomedical Engineering (Discontinued) 2.1 (2009): 58-64.

With various high-maneuverability MACEs developed, improving the autonomy level is the natural next step to shorten the duration of stomach screening, reduce the learning curve for surgeons, and lowering the user workloads, which enables endoscopists to concentrate more on diagnosis of abnormalities inside the stomach. Currently, most capsule endoscopes are focusing on employing artificial intelligence (AI) techniques in abnormalities diagnosis, which helps to lower the workloads of surgeons (Wu et al. “Real-time artificial intelligence for detecting focal lesions and diagnosing neoplasms of the stomach by white-light endoscopy (with videos).” Gastrointestinal Endoscopy 95.2 (2022): 269-280.). Nevertheless, AI-assisted diagnosis typically relies on a number of high-quality images of the stomach captured by the capsule endoscope. Yao et al. (Yao K. “The endoscopic diagnosis of early gastric cancer”. Ann Gastroenterol. 2013; 26(1): 11-22.) proposed a systematic screening protocol for the stomach, which claimed that in total 22 endoscopic images of different gastric sites could be used for mapping and diagnosing the entire stomach. And Xiao et. al. (Xiao et al. “Fully automated magnetically controlled capsule endoscopy for examination of the stomach and small bowel: a prospective, feasibility, two-centre study”. The Lancet Gastroenterology & Hepatology 6.11 (2021): 914-921.) reported an automatic stomach routine by using a MACE to detect and record 6 major parts of the stomach, including cardia, gastric fundus, gastric body, gastric antrum, gastric angle, and pylorus. However, their system lacks closed-loop control of the capsule endoscope, which makes the screening procedure not efficient and reliable enough. Therefore, by employing advanced control strategies and AI techniques, MACEs can be controlled to automatically detect these gastric sites and capture images from the optimal angle of view, which make it possible to achieve automatic and comprehensive stomach screening.

Hitherto, there is very few capsule endoscopes that can achieve automatic stomach screening based on AI-based gastric site detection and active motion control. It would be a significant advantage to introduce a capsule endoscope with active actuation and closed-loop motion control to fulfill such an automatic stomach screening routine, which can greatly shorten the duration of the screening procedure and reduce the workload of surgeons.

### SUMMARY OF THE INVENTION

The present invention introduces an automatic stomach screening method using a magnetically actuated capsule endoscope, which shortens the screening duration and reduces the surgeon workload while ensuring the accuracy and reliability of diagnostic results. The method disclosed herein, employs AI-based detection techniques and advanced closed-loop motion controllers.

In a first aspect of the present invention, an automatic screening method is composed of a well-planned workflow, which controls the capsule endoscope to subsequently view six major gastric parts for mapping the entire stomach. The method disclosed herein, uses a capsule endoscope for capturing images inside the stomach and an electromagnetic actuation system, composed of several coils and cores, for actuating the capsule endoscope. In one embodiment of the present invention, the present invention employs a fuzzy proportional-integral-derivative (PID) controller to actuate the capsule endoscope to move to different sites of stomach and uses Faster-RCNN, a well-known learning-based object detection benchmark model to detect gastric sites with specific characteristics. A proportional sliding mode visual servo controller is employed for orientation adjustment of the capsule endoscope to enable the capsule to capture images of the detected gastric sites from the optimal angle of view. A 6D magnetic localization strategy, based on the fusion of magnetic field measured by a Hall-effect sensor array and orientation data from the capsule's IMU, is utilized to estimate the 6D pose of the capsule to enable closed-loop motion of the capsule. The AI detection techniques, motion controllers, as well as the localization algorithm could be replaced by other methods that can achieve similar performance.

In one embodiment of the present invention, a capsule endoscope capsule endoscope consists of an annular permanent magnet, multiple printed circuit boards (PCBs) and a shell housing these components. More specifically, these PCBs include an image acquiring unit, an illumination unit, a data transmission unit, and an inertial measurement unit (IMU) for collecting 3D orientation information of the capsule. In one embodiment of the present invention, the capsule endoscope can wirelessly transmit video and IMU data to the external control unit, while the control unit can send commands to the capsule endoscope for adjusting data transmission frequency and lightness strength.

In one embodiment of the present invention, the magnetic actuation unit consists of multiple electromagnets, which distributed above and below to surround a workspace for holding the patient body. An individual electromagnet comprises a copper coil and an iron core. The configuration of the electromagnet array varies depending on different application scenarios, which surrounds a workspace that is sufficient for holding the patient body.

In one embodiment of the present invention, by applying specific currents to the electromagnets, desired magnetic fields can be generated at the position of the capsule endoscope, which results in magnetic forces and torques exerting the capsule and thus enable steering and translation of the capsule endoscope.

In another embodiment of the present invention, there is a Hall-effect sensor array located at the bottom of the workspace for 5D localization of the capsule endoscope, which thus enables closed-loop motion control of the capsule. The 6D pose of the capsule endoscope can be acquired by fusing the IMU data and the magnetic fields measured by the Hall-effect sensor array located at the bottom of the workspace.

In a second aspect of the present invention, the workflow of the present invention is described as follows. First, the patient needs to drink a lot of water before the screening procedure, which used to full the stomach and provide the buoyancy for the capsule endoscope. Next, the patient swallows the capsule endoscope and lies on the bed inside the actuation system. The actuation system generates magnetic fields to control translation and orientation of the capsule endoscope to capture images of six major parts of the stomach, with 6D pose of the capsule provided for closed-loop control. Six major parts of the stomach are subsequently detected using AI techniques, including cardia, gastric fundus, gastric body, gastric antrum, gastric angle, and pylorus. More than three images are captured for each part of the stomach, with visual servo control employed to adjust the orientation of the capsule to the optimal view of angle. Then, after sufficient are collected for images mapping the entire stomach, the surgeon can use the joysticks on the control unit to manually control the capsule endoscope to conduct detailed diagnosis on region of interest. Finally, the capsule endoscope is naturally eliminated from the human body under the peristalsis wave of the GI tract.

In a third aspect of the present invention, the magnetic actuation model of the actuation system is established to reveal the relationship between the applied currents to the electromagnets and the magnetic field (force or torque) at a specific position. The kinematic model of the capsule endoscope is analysed to enable visual servo control for orientation adjustment of the capsule based on the detected feature of the stomach. Moreover, a localization model is constructed to acquire the 6D pose of the capsule endoscope, which is based on the fusion of IMU data and magnetic fields measured by the Hall-effect sensor array.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a systematic flowchart showing a procedure that uses a magnetically actuated capsule endoscope to conduct automatic stomach screening under AI detection techniques and advanced closed-loop motion controllers.

FIG. 2 illustrates the system overview of the magnetically actuated capsule endoscope device for automatic stomach screening according to the features of the proposed invention, which includes: 1. capsule endoscope; 2. upper electromagnet module; 3. actuation system base; 4. patient bed; 5. lower electromagnet module; 6. Hall-effect sensor array; 7. doctor chair; 8. workbench; 9. first screen for displaying stomach view with AI detection; 10. stomach view with AI detection; 11. second screen for display raw stomach scene; 12. host computer.

FIG. 3 illustrates front view of capsule endoscope according to the features of the proposed invention, which includes: 1-1: Bluetooth (BLE); 1-2: inertial measurement unit (IMU); 1-3: annular permanent magnet; 1-4: shell; 1-5: camera module; 1-6: light-emitting diode; 1-7: analog video (AV) transmitter; 1-8: AV antenna; 1-9: power procession module; 1-10: battery.

FIG. 4 illustrates the actuation system base of the invented magnetically actuated capsule endoscope device according to the features of the present invention, which mainly includes 3-1: upper actuation mounting space for upper electromagnet module; 3-2: working space for patient bed and patient; 3-3: lower mounting space for lower electromagnet module and Hall-effect sensor array.

FIG. 5 (a)-(d) shows partial sectional views of the upper and lower electromagnet module of the invented magnetically actuated capsule endoscope device according to the features of the present invention, which consists of multiple electromagnets distributed in various configurations. In this embodiment, there are three to six electromagnets forming an upper and lower module. Each electromagnet contains: 5-1: copper coil; 5-2: pure iron core.

FIG. 6 illustrates the localization diagram of the invented magnetically actuated capsule endoscope device according to the features of the present invention. These mainly include 6-1: Hall-effect sensor mounting printed circuit board; 6-2: three-axis Hall-effect sensor; 13: the stomach environment.

FIG. 7 is the block diagram illustrating the capsule's pose estimation algorithm, which formulates and solves an optimization problem based on the fusion of magnetic fields measured by the Hall-effect sensor array and orientation information measured by the capsule's IMU.

FIG. 8 is a block diagram schematically showing an example of a fuzzy PID position controller with 6D capsule's pose feedback.

FIG. 9 illustrates the kinematic analysis of the capsule endoscope. (a) Mapping from the world frame to the capsule frame, with a magnetic moment vector m_{c} and a displacement vector r_{c} representing the pose of the capsule. m_{c} can be represented using two angles ν and φ in two orthogonal planes. (b) Coordinate frames of the capsule using Denavit-Hartenberg (DH) representation, with two virtual orthogonal revolute joints equipped in the capsule.

FIG. 10 illustrates the pinhole camera model for visual servo control. A point r(x,y,z) is projected to a point ρ(u,v) on the image plane.

FIG. 11 is a block diagram schematically showing the procedure of visual servo control of the capsule endoscope, which is to adjust the capsule's orientation to capture images from the optimal angle of view.

FIG. 12 illustrates an example of using magnetic field and force to control the capsule to move from a position to another desired position with orientation adjustment in the meantime. Such a process contains four operations: I: the start position in the stomach; II: the second position in the stomach; III: the third position in the stomach; IV: the end position in the stomach.

FIG. 13 illustrates examples of six major gastric parts detected by AI detection techniques, with (a): cardia of the stomach; (b): gastric fundus of the stomach; (c): gastric body of the stomach; (d): gastric antrum of the stomach; (e): gastric angle of the stomach; (f): pylorus of the stomach.

FIG. 14(a)-(f) illustrate the automatic stomach screening workflow using the invented magnetically actuated capsule endoscope device, which contains six major parts of the stomach: 13-1: cardia of the stomach; 13-2: gastric fundus of the stomach; 13-3: gastric body of the stomach; 13-4: gastric antrum of the stomach; 13-5: gastric angle of the stomach; 13-6: pylorus of the stomach. Capsule endoscope can perform the rotation and translation to specific area of stomach and execute the screening and transmission image to information processing module according to the features of the present invention.

FIG. 15 illustrates experiment setup by using nine electromagnets actuating a capsule endoscope. (a) Experimental setup of stomach automatic detection. (b) Capsule visual servo control process. (c) Capsule self-circle track process.

FIG. 16 illustrates experimental results of visual servo control. (a) Pixel trajectories of the target. (b) Corresponding pixel errors.

FIG. 17 illustrates experimental setup and process of the automatic stomach screening test. (a) The top view of the experiment setup. (b) The side view of experiment setup. (c) The views of four makers. (d) Four detected target features.

FIG. 18 illustrates experimental results of the automatic stomach screening benchtop test. (a) Capsule position control results. (b) Pixel errors of tracking four target features.

### DETAILED DESCRIPTION OF THE PREFERRED EMBODIMENTS

Exemplary embodiments of a capsule endoscope according to the present invention are explained in detail below with reference to the accompanying drawings. The present invention is not limited to the embodiments.

This invention provides a system for imaging one or more specific structures in a stomach within a workspace. In one embodiment, said system comprises: a) An active capsule endoscope to be placed into said stomach, comprising a camera, an inertial measurement unit and a permanent magnet; b) An actuation unit comprising a Hall-effect sensor array located at bottom of said workspace and a plurality of magnetic generators for generating a desired magnetic field; c) A controller unit comprising an image module, a closed-loop position controller, and a vision-based servo controller; said controller unit obtains images from said camera and orientation data from said inertial measurement unit; said controller unit obtains magnetic field data on said desired magnetic field from said Hall-effect sensor array; said controller unit generates a control input to said actuation unit to drive said active capsule endoscope along a pre-planned trajectory in said stomach; wherein said controller unit calculates in real-time a 6D-pose of said active capsule endoscope, χ, using said orientation data and said magnetic field data; said 6D-pose, χ, is feedback to said closed-loop position controller to perform translation and orientation adjustment by controlling said plurality of magnetic generators so as to remain in said pre-planned trajectory; said vision-based servo controller controls said plurality of magnetic generators based on said images and said 6-D pose, χ, so as to actuate said active capsule endoscope to image said one or more specific structures, wherein said image module matches said images against a database of stomach landmarks and general stomach three-dimensional shape to locate and image said one or more specific structures.

In one embodiment, said image module matches said images using AI.

In one embodiment, said system further comprising a dynamic trajectory generator for guiding said active capsule endoscope along said pre-planned trajectory.

In one embodiment, said active capsule endoscope further comprises one or more of an illumination unit or a data transmission unit.

In one embodiment, said Hall-effect sensor array comprises one or more three-axis hall sensors distributed in three-dimensional space of said workspace according to said desired magnetic field.

In one embodiment, each of said one or more three-axis hall sensors are oriented differently with respect to orthogonal coordinates (X, Y, Z) of said desired magnetic field.

In one embodiment, said controller unit comprises an algorithm for calculating said 6D-pose of said active capsule endoscope, χ; said algorithm comprises the steps of: a) Measuring magnetic field B_{cap} generated by said permanent magnet using said Hall-effect sensor array and representing as a stacked matrix form B_{H}:

$${B}_{H} {=}^{Δ} [ \begin{matrix}{B}_{H}^{11} & ⋯ & {B}_{H}^{1 j} \\ ⋮ & ⋱ & ⋮ \\ {B}_{H}^{i 1} & ⋯ & {B}_{H}^{ij}\end{matrix} ] i , j ∈ {ℕ}_{+}$$

b) Modelling magnetic field in each Hall-effect sensor in said Hall-effect sensor array as a stacked matrix form B_{c}(χ,Pᵢⱼ) which relative to said 6D-pose, χ, and said position of ij Hall-effect sensor Pᵢⱼ:

$${B}_{c} ( χ , {P}_{ij} ) {=}^{Δ} [ \begin{matrix}{B}_{c} ( χ , {P}_{11} ) & ⋯ & {B}_{c} ( χ , {P}_{1 j} ) \\ ⋮ & ⋱ & ⋮ \\ {B}_{c} ( χ , {P}_{i 1} ) & ⋯ & {B}_{c} ( χ , {P}_{ij} )\end{matrix} ]$$

c) Measuring orientation of said active capsule endoscope, φ_{I}, θ_{I}, γ_{I}, with respect to a world frame by a filter algorithm; d) Calculating said 6-DOF pose, χ, by solving an optimization function

$$χ = \begin{matrix}arg min \\ χ\end{matrix} ‖ {B}_{H} - {B}_{c} ( χ , {P}_{ij} ) ‖$$

constructed under the condition where φ≡φ_{I}, θ≡θ_{I}, γ≡γ_{I}.

In one embodiment, B_{c}(χ, Pᵢⱼ) is selected from a model selected from the group consisting of magnetic dipole model, magnetic multipole model, and fitting model of magnetic field in mathematical paradigm.

In one embodiment, said filter algorithm of step (c) is selected from the group consisting of Kalman filter, extended Kalman filter, and complementary filter.

In one embodiment, each of said plurality of magnetic generators is controlled by an input current to generate said desired magnetic field, wherein, at a given position, p, in said desired magnetic field, B, a magnetic force, F, is exerted on said active capsule endoscope based on said input current, c, according to the formula:

$$[ \begin{matrix}B \\ F\end{matrix} ] = [ \begin{matrix}𝔹 ( p ) \\ {m}^{T} {𝔅}_{x} ( p ) \\ {m}^{T} {𝔅}_{y} ( p ) \\ {m}^{T} {𝔅}_{z} ( p )\end{matrix} ] [ \begin{matrix}{C}_{1} \\ ⋮ \\ {C}_{n}\end{matrix} ] = {𝒜}_{B , F} ( m , p ) c ,$$

wherein m is the magnetic moment of said permanent magnet.

In one embodiment, said closed-loop position controller or said vision-based servo controller is a controller selected from the group consisting of a proportional-integral-derivative controller, fuzzy controller, adaptive controller, sliding mode controller, and model predictive controller.

In one embodiment, said control input is formulated based on kinematic analysis of said active capsule endoscope using:

$$R [ \begin{matrix}\dot{{s}_{x}} \\ \dot{{s}_{y}}\end{matrix} ] = {Q}_{system} [ \begin{matrix}\dot{ϑ} \\ \dot{φ}\end{matrix} ] with {Q}_{system} = {Q}_{image} [ \begin{matrix}{R}_{C}^{{W}_{T}} & O \\ O & {R}_{C}^{{W}_{T}}\end{matrix} ] {Q}_{capsule} ,$$

wherein Q_{capsule} is Jacobian matrix of the capsule; Q_{system} is Jacobian matrix of said system; ν and φ are the heading angles of said active capsule endoscope on the horizontal and vertical plane correspondingly; R is a 2×2 rotation matrix related to the rotational angle around said active capsule endoscope's heading;

$${R}_{C}^{W}$$

is a 3×3 rotation matrix from a world coordinate frame to a camera frame.

In one embodiment, said stomach landmarks comprise one or more of cardia, gastric fundus, gastric body, gastric antrum, gastric angle, and pylorus.

In one embodiment, said pre-planned trajectory comprises a sequence from cardia, gastric fundus, gastric body, gastric antrum, gastric angle, to pylorus.

This invention also provides a method for imaging one or more specific structures in a stomach using the system of this invention. In one embodiment, said method comprises the steps of: a) Introducing said active capsule endoscope into said stomach; b) Sending instructions from said controller unit to said actuation unit to generate said desired magnetic field to move said active capsule endoscope according to said pre-planned trajectory; and c) Capturing one or more images with said active capsule endoscope while moving along said pre-planned trajectory within said stomach.

In one embodiment, said instructions of step (b) are generated by a method comprising the steps of: i) Identifying a first location of said active capsule endoscope by analyzing one or more images captured by said active capsule endoscope using said image module; ii) Mapping said one or more images to stomach landmarks; iii) Obtaining current 6D-pose of said active capsule endoscope; iv) Calculating discrepancy between said current 6D-pose and said pre-planned trajectory; and v) Generating instructions for a desired magnetic field to move said active capsule endoscope towards said pre-planned trajectory.

In one embodiment, said stomach is fluid-filled.

In one embodiment, said pre-planned trajectory follows a sequence from cardia, gastric fundus, gastric body, gastric antrum, gastric angle, to pylorus.

In one embodiment, said method further comprises eliminating said active capsule endoscope from said subject using natural peristalsis of the gastrointestinal tract after step (c).

In one embodiment, said method further comprises controlling said active capsule endoscope manually to inspect a region of interest after step (c).

An automatic stomach screening method disclosed in the present invention comprises a capsule endoscope, which is swallowed by the patient and conducts screening inside the stomach with closed-loop magnetic actuation. With the proposed routine, this device can automatically undertake magnetic levitation and move to specific positions to detect gastric parts with different characteristics under advanced position controllers and AI techniques, and then capture sufficient images of these gastric parts in the optimal angle of view under the visual servo controller. During the whole procedure, 6-DOF pose of the capsule endoscope is acquired by a localization method based on fusion of orientation data from the embedded IMU and magnetic field data from the Hall-effect sensor array, which thus enables closed-loop motion control of the capsule endoscope and ensures the accuracy and reliability of the stomach screening results. This automatic routine is aimed to shorten the duration of stomach screening procedure. In addition, the major task of surgeons is to perform supervision and diagnosis of stomach abnormalities, and thus lowers the workloads of surgeons.

Main advantages of the present invention using a magnetically actuated capsule endoscope to conduct automatic stomach screening includes: (1) Automatic routine shortens the duration of stomach screening procedure and lowers the workloads of surgeons while ensuring the accuracy and reliability of the screening results; (2) Employment of AI techniques enables the capsule endoscope to detect different gastric sites that maps the entire stomach and assists surgeons to diagnose the stomach abnormalities; (3) 6-DOF pose feedback enables the closed-loop motion control of the capsule endoscope, which improves the efficiency of screening process and accuracy of screening results. (4) Advanced controllers designed for capsule's position and orientation adjustment enables the capsule endoscope screens specific sites of the stomach efficiently and stably.

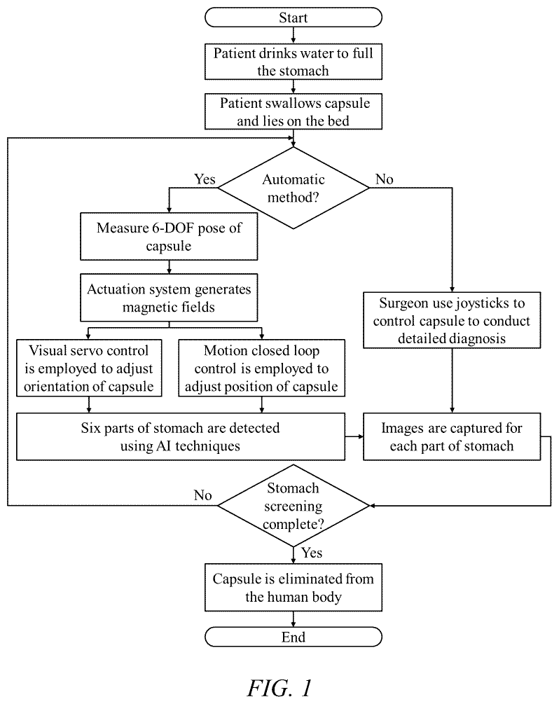

FIG. 1 illustrates a systematic flowchart showing the procedure of automatic stomach screening using a capsule endoscope under closed-loop magnetic actuation. The workflow of the present invention is as follows: First, the patient needs to drink a lot of water before the screening procedure, which used to full the stomach and provide the buoyancy for the capsule endoscope. Next, the patient swallows the capsule endoscope and lies on the bed inside the actuation system. With the automatic screening routine selected, the 6D pose of the capsule endoscope is acquired based on the data fusion of the magnetic field measured by the Hall-effect sensor array and the orientation information obtained from the capsule's IMU. The actuation system generates magnetic fields to control translation and orientation of the capsule endoscope, which enables the capsule subsequently capture images of six major parts of the stomach that are detected by the AI detection techniques. Six major parts of the stomach are captured subsequently, including cardia, gastric fundus, gastric body, gastric antrum, gastric angle, and pylorus. More than three images are captured for each part of the stomach, with visual servo control employed to adjust the orientation of the capsule to the optimal view of angle. Then, after sufficient are collected for images mapping the entire stomach, the surgeon can use the joysticks on the control unit to manually control the capsule endoscope to conduct detailed diagnosis on region of interest. Finally, the capsule endoscope is naturally eliminated from the human body under the peristalsis wave of the GI tract.

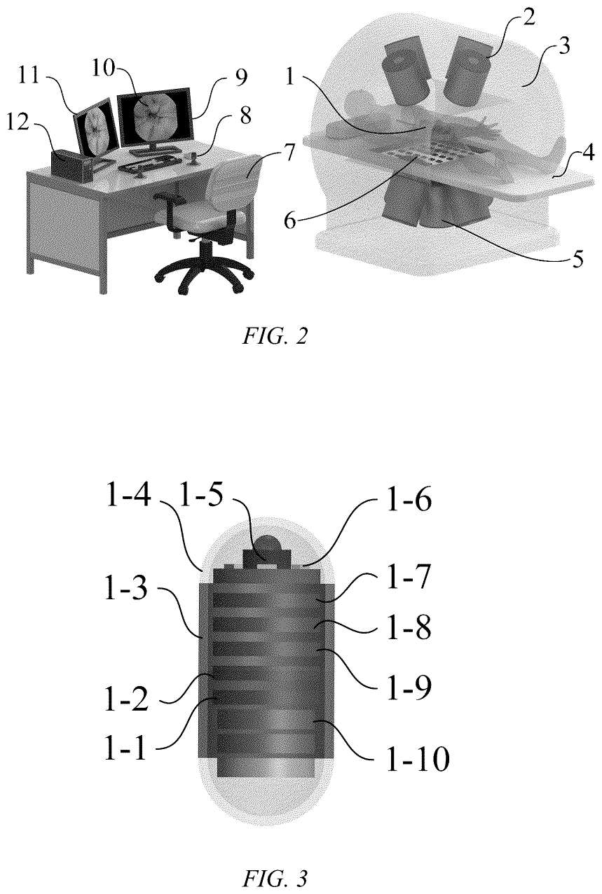

Referring to FIG. 2, a preferred embodiment of the magnetically actuated capsule endoscope device is illustrated according to the invention. There are three subassemblies shown in the device: the control system, the actuation system, and the capsule endoscope 1. The control unit consists of a host computer 12, two display screen 9 and 11 showing the detected scene 10 inside the stomach, a chair 7, and a bench 8. The actuation unit is composed of a base 3, an upper electromagnet module 2, a patient bed 4, a lower electromagnet module, and a Hall-effect sensor array 6. The control unit is used for processing data (image and IMU data from the capsule endoscope, magnetic field data from the Hall-effect sensor array) and running algorithms (e.g., magnetic actuation algorithm, localization algorithm, and AI techniques), while the actuation unit is designed for generating magnetic fields to control the motion of the capsule endoscope and collecting data to provide pose feedback of the capsule.

As illustrated in FIG. 3, the capsule endoscope is designed for real-time screening and conducting diagnosis inside the stomach under magnetic actuation. The capsule endoscope mainly consists of an annular permanent magnet, several PCBs, and a shell housing these components. Typically, these PCBs include a camera module, an illumination module, a video transmission module, and a battery module. An IMU module is embedded inside the capsule for acquiring its orientation information, with the orientation data transmitted to external devices through Bluetooth techniques.

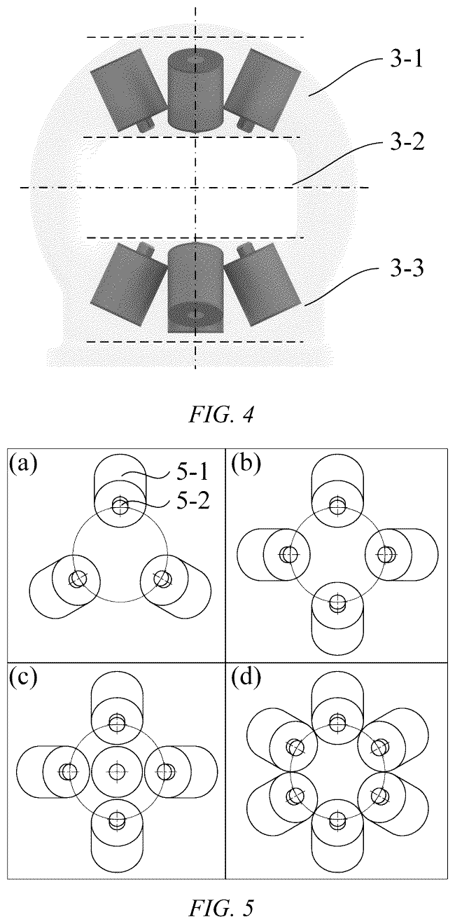

FIG. 4 illustrates the electromagnet distribution of the actuation system. There is a cuboid workspace 3-2 surrounded by the upper electromagnet module 3-1 and the lower electromagnet module 3-3, which is used for holding the patient body and conduct screening under the generated magnetic fields.

FIG. 5(a)-(d) illustrate some examples of the configuration of the upper or lower electromagnet module. This configuration varies depending on the application requirements. Each electromagnet has a copper coil and a pure iron core, with a cylindrical shape. With a given static configuration of electromagnets, each electromagnet generates a magnetic field throughout the workspace that can be precomputed. At any given position p, the magnetic field generated by a given electromagnet can be expressed by Bₑ(p)=\hat{B}ₑ(p)cₑ, with cₑ as the current through the electromagnet. Alternatively, the magnetic field generated by a given electromagnet can also calculated using the magnetic dipole or multipole model. The superimposed magnetic field at a given position p generated by multiple electromagnets can be calculated by linear summation for simplicity, which can be expressed as

$$B ( p ) = {\sum }_{e = 1}^{n} {B}_{e} ( p ) = {\sum }_{e = 1}^{n} {\hat{B}}_{e} ( p ) {c}_{e} = [ \begin{matrix}{\hat{B}}_{1} ( p ) & ⋯ & {\hat{B}}_{n} ( p )\end{matrix} ] [ \begin{matrix}{C}_{1} \\ ⋮ \\ {C}_{n}\end{matrix} ] = 𝔹 ( p ) c$$

The magnetic force exerted on the capsule endoscope is related to the magnetic field gradient, which can be expressed as

$$F = {[ \begin{matrix}\frac{\partial B}{\partial x} & \frac{\partial B}{\partial y} & \frac{\partial B}{\partial z}\end{matrix} ]}^{T} m ,$$

with m as the magnetic moment of the permanent magnet embedded in the capsule endoscope. More specifically,

$$\frac{\partial B ( p )}{\partial x} = [ \begin{matrix}\frac{\partial {\hat{B}}_{1} ( p )}{\partial x} & … & \frac{\partial {\hat{B}}_{n} ( p )}{\partial x}\end{matrix} ] [ \begin{matrix}{C}_{1} \\ ⋮ \\ {C}_{n}\end{matrix} ] = {\text{?}}_{x} ( p ) c$$

$$\frac{\partial B ( p )}{\partial y} = [ \begin{matrix}\frac{\partial {\hat{B}}_{1} ( p )}{\partial y} & … & \frac{\partial {\hat{B}}_{n} ( p )}{\partial y}\end{matrix} ] [ \begin{matrix}{C}_{1} \\ ⋮ \\ {C}_{n}\end{matrix} ] = {\text{?}}_{y} ( p ) c$$

$$\frac{\partial B ( p )}{\partial z} = [ \begin{matrix}\frac{\partial {\hat{B}}_{1} ( p )}{\partial z} & … & \frac{\partial {\hat{B}}_{n} ( p )}{\partial z}\end{matrix} ] [ \begin{matrix}{C}_{1} \\ ⋮ \\ {C}_{n}\end{matrix} ] = {\text{?}}_{z} ( p ) c$$

It is assumed that there is no constraint for the capsule endoscope inside the stomach full of liquid, thus the capsule's direction (can also be represented by m) can be considered to be always aligned to the direction of the magnetic field B(p). Therefore, by reformulating the equations mentioned above, the magnetic field and force on the capsule endoscope can be expressed as follows:

$$[ \begin{matrix}B \\ F\end{matrix} ] = [ \begin{matrix}𝔹 ( p ) \\ {m}^{T} {\text{?}}_{x} ( p ) \\ {m}^{T} {\text{?}}_{y} ( p ) \\ {m}^{T} {\text{?}}_{z} ( p )\end{matrix} ] [ \begin{matrix}{C}_{1} \\ ⋮ \\ {C}_{n}\end{matrix} ] = {𝒜}_{B , F} ( m , p ) c$$

For a desired magnetic field and force, the currents applied to the electromagnets can be calculated by using the pseudoinverse:

$$c = {{𝒜}_{B , F} ( m , p )}^{†} [ \begin{matrix}{B}_{des} \\ {F}_{des}\end{matrix} ]$$

Consequently, the position and orientation of the capsule endoscope can be actively controlled by changing the currents applied to the electromagnets and thus changing the magnetic field and force exerted on the capsule endoscope.

FIG. 6 illustrates the localization diagram of the invented magnetically actuated capsule endoscope device. The coordinates are established as follows: orthogonal coordinate (X,Y,Z) is defined in the Hall-effect sensor mounting printed circuit board with the origin on board, which is named H-frame; orthogonal coordinate (x, y, z) is defined in capsule endoscope with the origin on geometric centre of capsule endoscope, which is referred to as C-frame; orthogonal coordinate (Xᵢⱼ, Yᵢⱼ, Zᵢⱼ) are defined in ij Hall-effect sensor with the origin on the geometric centre of ij Hall-effect sensor, where i represents row and j stands for column in the array (i,j∈ [symbol] ).

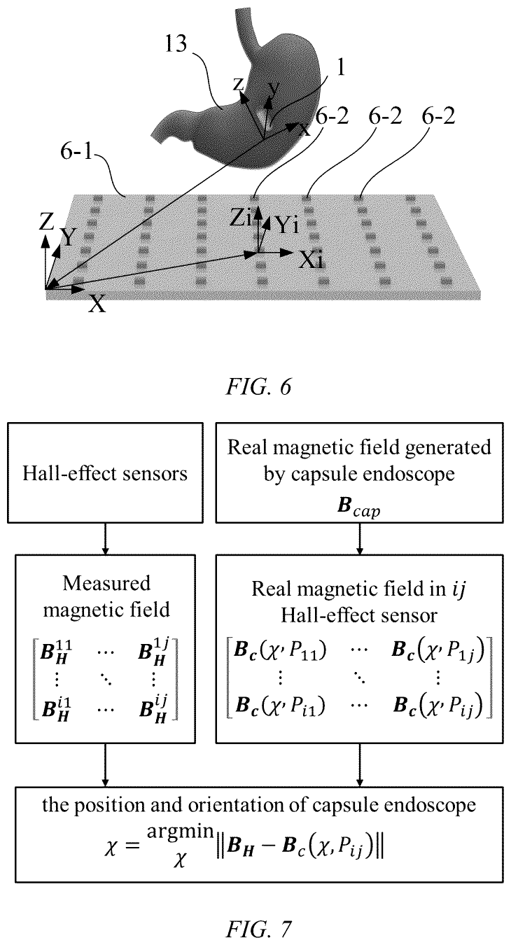

FIG. 7 illustrates an example of 6D capsule's pose estimation method by formulating and solving an optimization problem. According to an aspect of the invention the localization system measures the six degrees of freedom (position x,y,z and orientation φ,θ,γ with respect to H-frame) of capsule endoscope. For brevity, the definition χ≅[x y z φ θ γ] is derived. It is another aspect of the invention that when capsule endoscope works in stomach environment, the permanent magnet inside capsule endoscope generates magnetic field B_{cap} which is measured partially by Hall-effect sensors. The measurement results can be expressed in a stacked matrix form as

$${B}_{H} {=}^{Δ} [ \begin{matrix}{B}_{H}^{11} & ⋯ & {B}_{H}^{1 j} \\ ⋮ & ⋱ & ⋮ \\ {B}_{H}^{i 1} & ⋯ & {B}_{H}^{ij}\end{matrix} ] i , j ∈ {ℕ}_{+}$$

$$where {B}_{H}^{ij}$$

is the measured magnetic field in ij Hall-effect sensor.
On the other hand, the modeled magnetic field in each Hall-effect sensor can be expressed as

$${B}_{c} ( χ , {P}_{ij} ) {=}^{Δ} [ \begin{matrix}{B}_{c} ( χ , {P}_{11} ) & ⋯ & {B}_{c} ( χ , {P}_{1 j} ) \\ ⋮⋮ & ⋱ & ⋮ \\ {B}_{c} ( χ , {P}_{i 1} ) & ⋯ & {B}_{c} ( χ , {P}_{ij} )\end{matrix} ]$$

where Pᵢⱼ is the position of ij Hall-effect sensor with respect to H-frame, and

$${B}_{c}^{ij} ( χ , {P}_{ij} )$$

is the real magnetic field in ij Hall-effect sensor generated by capsule,

$${B}_{c}^{ij} ( χ , {P}_{ij} ) ⊂ {B}_{cap} .$$

It is noted that B_{c}(χ,Pᵢⱼ) is related to position and orientation of capsule endoscope and the relative position between capsule endoscope and ij Hall-effect sensor.
It is worth noting that B_{c}(χ,Pᵢⱼ) could be represented by means of certain models, such as magnetic dipole model, magnetic multipole model, fitting model of magnetic field in mathematical paradigm. In addition, there is an IMU inside capsule endoscope measuring the orientation φ_{I},θ_{I},γ_{I} with respect to H-frame in real time by means of filter algorithm, like Kalman filter, extended Kalman filter, and complementary filter and so on. Another aspect of the invention is that the position and orientation of capsule endoscope can be calculated by constructing and solving an optimization function

$$χ = \begin{matrix}arg min \\ χ\end{matrix} ‖ {B}_{H} - {B}_{C} ( χ , {P}_{ij} ) ‖$$

where φ≡φ_{I}, θ≡θ_{I}, γ ≡γ_{I}. Consequently, the position and orientation of the capsule endoscope can be obtained.

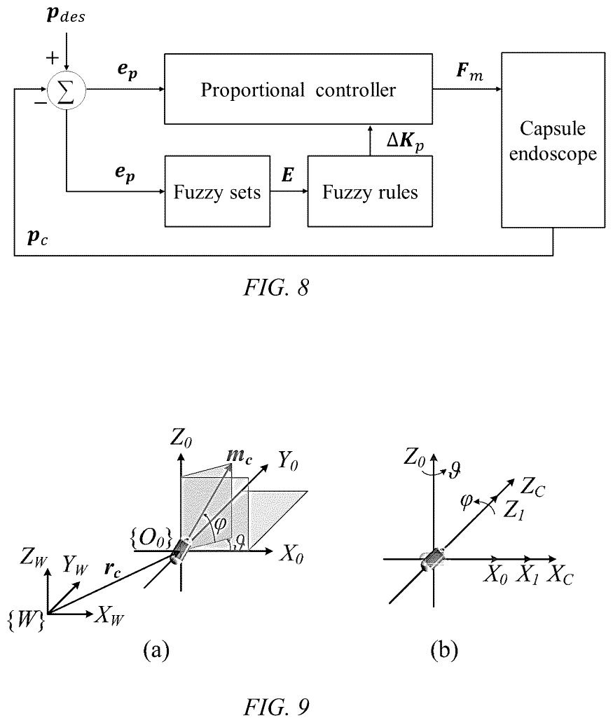

FIG. 8 is a block diagram showing an example of the position controller using fuzzy logics. As an embodiments of the present invention, a fuzzy proportional method is designed for controlling the translation and levitation of the capsule endoscope. Firstly, the position error is defined as the difference between the desire position and the current position: eₚ=p_{des}−p_{c}. Subsequently, e is transformed into a discrete unit to obtain an input E of the fuzzy controller. Finally, E are converted from the system-defined fuzzy rules into the corrections ΔKₚ for the parameters of the proportional controller, where the fuzzy sets of the fuzzy variables (E and ΔKₚ) are determined by the triangular membership function. Therefore, the magnetic force is calculated as

$${F}_{m} = {K}_{p} {e}_{p} with {K}_{p} = {K}_{p} + Δ {K}_{p}$$

During the translation control, the orientation of the capsule endoscope is assumed to be aligned with the direction of the applied magnetic field Bₘ. Therefore, the currents applied to the electromagnets can be calculated based on the desired magnetic field and force.

FIG. 9 (a) illustrates the mapping from the world frame to the capsule frame, with a magnetic moment vector m_{c} and a displacement vector r_{c} representing the pose of the capsule. m_{c} can be represented using two angles ν and φ in two orthogonal planes.

FIG. 9 (b) illustrates the Denavit-Hartenberg (DH) representation of the capsule's coordinate frames. By treating the capsule as a mechanism with two orthogonal revolute joints, the kinematics of the capsule endoscope can be analysed by using three coordinate frames defined. The associated transformation matrices are

$${T}_{1}^{0} = [ \begin{matrix}{c}_{ϑ} & 0 & - {s}_{ϑ} & 0 \\ {s}_{ϑ} & 0 & {c}_{ϑ} & 0 \\ 0 & - 1 & 0 & 0 \\ 0 & 0 & 0 & 1\end{matrix} ] and {T}_{C}^{1} = [ \begin{matrix}{c}_{ϑ} & 0 & - {s}_{ϑ} & 0 \\ {s}_{ϑ} & 0 & {c}_{ϑ} & 0 \\ 0 & - 1 & 0 & 0 \\ 0 & 0 & 0 & 1\end{matrix} ]$$

Where s_{ν}=sin ν, c_{ν}=cos ν, s_{φ}=sin φ, and c_{φ}=cos φ. Therefore, the pose of camera frame with respect to the world frame is depicted by the following transformation matrix:

$${T}_{C}^{W} = {T}_{0}^{W} \cdot {T}_{1}^{0} \cdot {T}_{C}^{1} = [ \begin{matrix}{R}_{C}^{W} & {P}_{C}^{W} \\ 0 & 1\end{matrix} ] ∈ {ℝ}^{4 \times 4} with {T}_{C}^{W} = [ \begin{matrix}1 & 0 & 0 & {r}_{x} \\ 0 & 1 & 0 & {r}_{y} \\ 0 & 0 & 1 & {r}_{z} \\ 0 & 0 & 0 & 1\end{matrix} ]$$

are the rotation matrix and the position vector, respectively. If the linear and angular velocities of the camera frame are separately defined in the world frame, then one can naturally obtain the velocity level kinematic equation:

$$[ \begin{matrix}{υ}_{C}^{W} \\ {ω}_{C}^{W}\end{matrix} ] = {J}_{c a p s u l e} [ \begin{matrix}\dot{ϑ} \\ \dot{φ}\end{matrix} ] where {υ}_{C}^{W} and {ω}_{C}^{W}$$

are the linear and angular velocity of the camera frame, respectively; J_{capsule} is the Jacobian matrix of the capsule.

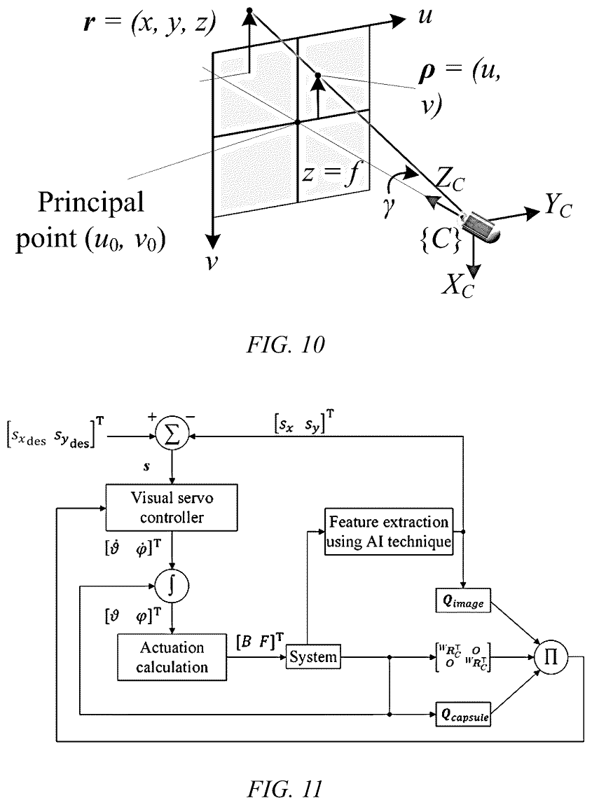

FIG. 10 illustrates the pinhole camera model for visual servo control. A point r(x,y,z) is projected to a point ρ(u,v) on the image plane. The rotation around the principal axis of the capsule (i.e., the magnetization direction of the embedded magnet) cannot be controlled by the magnetic actuation but may occur during the orientation control of the capsule, thus this rotation angle γ needs to be considered during visual servo modelling. The velocity of point ρ (u,v) on the image plane and the instantaneous linear and angular velocities (i.e.,

$${υ}_{C}^{W} and {ω}_{C}^{W}$$

of the camera frame yield the following mapping:

$$R [ \begin{matrix}\dot{u} \\ \dot{v}\end{matrix} ] = {J}_{i m a g e} [ \begin{matrix}{υ}_{C}^{C} \\ {ω}_{C}^{C}\end{matrix} ] with R = [ \begin{matrix}{C}_{γ} & - {S}_{γ} \\ {S}_{γ} & {C}_{γ}\end{matrix} ]$$

where γ is the rotation angle around the principal axis of the capsule measured by the embedded IMU, and the image Jacobian matrix:

$${J}_{image} = [ \begin{matrix}− \frac{\overline{f}}{z} & 0 & \frac{\overline{u}}{z} & \frac{\overline{u} \overline{v}}{f} & − \frac{\overline{f} + {\overline{u}}^{2}}{f} & \overline{v} \\ 0 & − \frac{\overline{f}}{z} & \frac{\overline{v}}{z} & \frac{{\overline{f}}^{2} + {\overline{v}}^{2}}{f} & − \frac{\overline{u} \overline{v}}{f} & − \overline{u}\end{matrix} ]$$

with ū=u−u₀, v̅=v−v₀, and f̅=f/ρ. Here, f and ρ are the focal length of the camera and the pixel width of each square pixel of an image, respectively. Depth z can be assumed constant due to the inherent error tolerance of visual servoing.
Note that

$${υ}_{C}^{C} and {ω}_{C}^{C}$$

are expressed in the camera frame, consequently, it finally yields:

$$R [ \begin{matrix}\dot{u} \\ \dot{v}\end{matrix} ] = {J}_{s y s t e m} [ \begin{matrix}\dot{ϑ} \\ \dot{φ}\end{matrix} ] with {J}_{system} = {J}_{i m a g e} [ \begin{matrix}{R}_{C}^{{W}_{T}} & O \\ O & {R}_{C}^{{W}_{T}}\end{matrix} ] {J}_{c a p s u l e}$$

where J_{system} is the Jacobian matrix of the whole robotic endoscope system.

FIG. 11 is a block diagram showing the process of employing visual servo control to adjust the orientation of the capsule endoscope so as to enable the capsule capture gastric images from the optimal view of angle. As an embodiment of the present invention, a sliding mode controller is employed for visual servo control of the capsule endoscope based on the detected gastric landmarks. Firstly, a sliding mode surface is defined to be the pixel position error on the image plane as

$$s = {e}_{ρ} = [ \begin{matrix}{u}_{c} - {u}_{des} \\ {v}_{c} - {v}_{des}\end{matrix} ]$$

where (u,v) and (u_{des},v_{des}) are the current and desired position of the target feature on the image plane, respectively. Then, its derivative is obtained as \dot{s}=ė. By using the exponential reaching law, one can obtained the control input as

$$[ \begin{matrix}\dot{ϑ} \\ \dot{φ}\end{matrix} ] = {J}_{s y s t e m}^{†} R ( - E sgn ( s ) - Ks ) where {J}_{s y s t e m}^{†}$$

is the pseudo inverse of the Jacobian matrix J_{system}. E=diag(εᵤ,εᵥ) and K=diag(κᵤ,κᵥ) are two diagonal positive matrices for adjusting the convergence speed. By integrating the resultant velocities \dot{ν} and \dot{φ}, one can calculate the desired magnetic field and force as

$$B = b = [ \begin{matrix}{C}_{ϑ} {C}_{φ} \\ {S}_{ϑ} {C}_{φ} \\ {S}_{φ}\end{matrix} ] and F = [ \begin{matrix}0 \\ 0 \\ {f}_{c}\end{matrix} ]$$

where b is the magnitude of the magnetic field and f_{c} is the magnetic force for compensating the gravity of the capsule endoscope. Therefore, with the desired magnetic field and force, one can calculate the currents applied to the electromagnets.

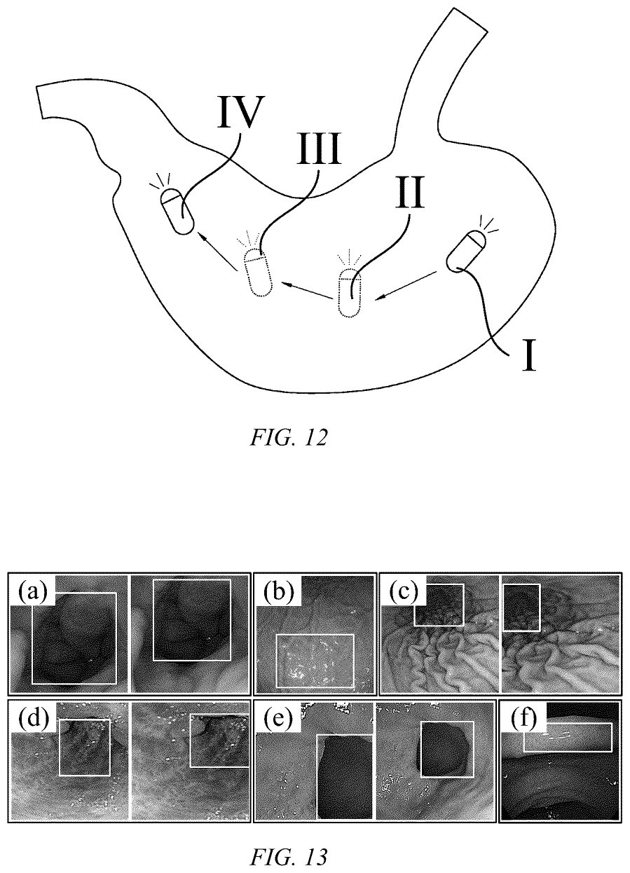

FIG. 12 illustrates an example of controlling the capsule endoscope to move from a position to another desired position under magnetic field and force. During the movement, desired magnetic fields and forces are calculated in real time based on 6D pose feedback of the capsule, which enable the capsule to perform translation and orientation adjustment.

FIG. 13(a)-(f) demonstrate some examples of six major gastric parts using AI detection techniques. As an embodiment of the present invention, Faster-RCNN, a well-known learning-based object detection benchmark model, is utilized here to detect gastric sites with specific characteristics. Such a detection model can be replaced by other similar detection techniques.

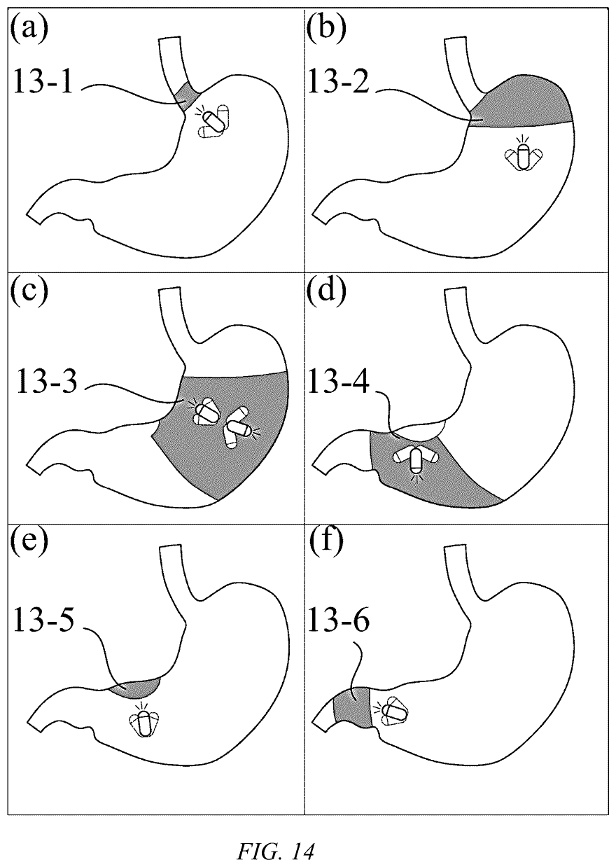

FIG. 14(a)-(f) illustrates the process of automatic stomach screening using the magnetically actuated capsule endoscope. In total six major parts of the stomach are screened for mapping the entire stomach, with the screening sequence as cardia (a)→gastric fundus (b)→gastric body (c)→gastric antrum (d)→gastric angle (e)→pylorus (f). AI techniques are utilized here for detecting these gastric landmarks as well as stomach abnormalities (e.g., polyp and ulcer). Advanced position controllers are employed here for controlling the translation of the capsule endoscope so that the capsule can move different pre-planned position to conduct diagnosis. Visual servo control is also employed here for orientation adjustment of the capsule endoscope to enable the capsule capture images of these gastric landmarks from the optimal angle of view. More than four images are captured for each landmark to map the entire stomach.

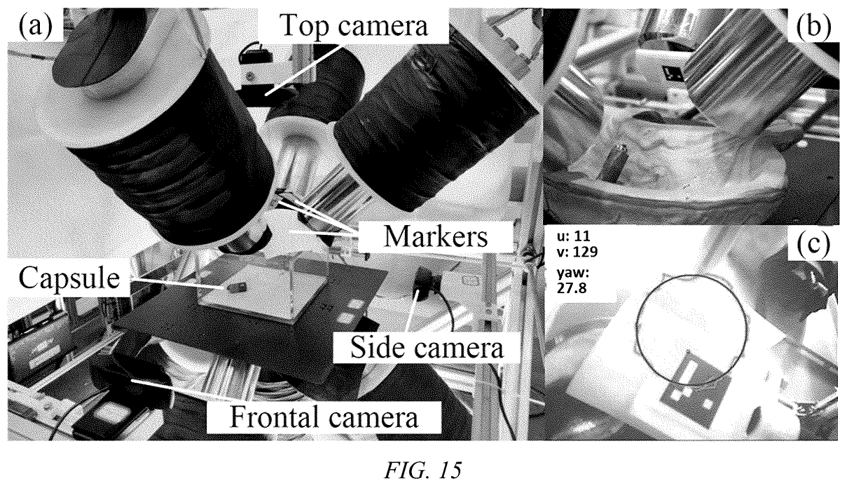

FIG. 15(a)-(c) illustrate experimental setup of an embodiment of the present invention that use nine electromagnets to actuate a capsule endoscope prototype to perform automatic stomach screening, with (a) overview of the system including an electromagnet array, a capsule endoscope and other related devices; (b) close view of the capsule endoscope lying inside a stomach phantom with magnetic actuation; (c) camera view of the capsule endoscope that is detecting a marker as a target feature.

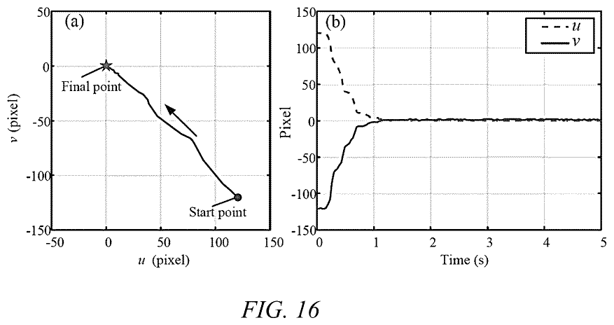

FIG. 16(a)-(b) illustrate experimental results of visual servoing using the prototypes and controllers mentioned in above embodiments, which indicating the capsule endoscope can track a target feature to a desired position on the image plane. (a) Pixel trajectories of the target. (b) Corresponding pixel errors.

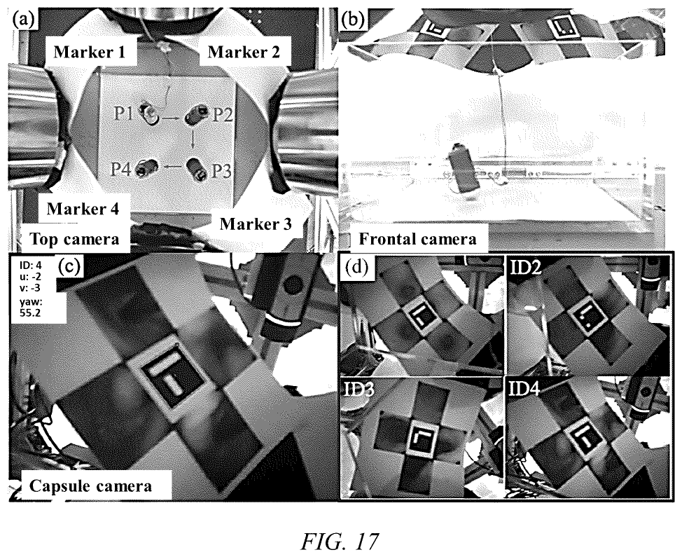

FIG. 17(a)-(d) demonstrates some images captured during the automatic stomach screening test that controls the capsule endoscope to move to several desired positions to detect specific features and capture their images from the optimal angle of view by employing a position controller and a visual servo controller. (a) The top view of the experiment setup. (b) The side view of experiment setup. (c) Camera view of the capsule endoscope. (d) Four detected target features.

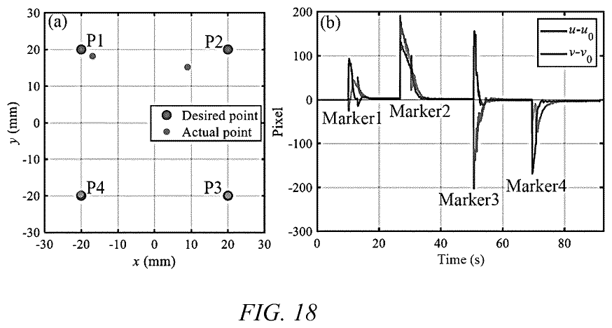

FIG. 18(a)-(b) illustrate experimental results of automatic stomach screening benchtop test. (a) Capsule position control results. (b) Pixel errors of tracking four target features.

What is claimed is:

1. A system for imaging one or more specific structures in a stomach within a workspace, said system comprises:
a. An active capsule endoscope to be placed into said stomach, comprising a camera, an inertial measurement unit and a permanent magnet;
b. An actuation unit comprising a Hall-effect sensor array located at bottom of said workspace and a plurality of magnetic generators for generating a desired magnetic field;
c. A controller unit comprising an image module, a closed-loop position controller, and a vision-based servo controller; said controller unit obtains images from said camera and orientation data from said inertial measurement unit; said controller unit obtains magnetic field data on said desired magnetic field from said Hall-effect sensor array; said controller unit generates a control input to said actuation unit to drive said active capsule endoscope along a pre-planned trajectory in said stomach;
wherein said controller unit calculates in real-time a 6D-pose of said active capsule endoscope, χ, using said orientation data and said magnetic field data; said 6D-pose, χ, is feedback to said closed-loop position controller to perform translation and orientation adjustment by controlling said plurality of magnetic generators so as to remain in said pre-planned trajectory;
said vision-based servo controller controls said plurality of magnetic generators based on said images and said 6-D pose, χ, so as to actuate said active capsule endoscope to image said one or more specific structures, wherein said image module matches said images against a database of stomach landmarks and general stomach three-dimensional shape to locate and image said one or more specific structures.

2. The system of claim 1, wherein said image module matches said images using AI.

3. The system of claim 1, further comprising a dynamic trajectory generator for guiding said active capsule endoscope along said pre-planned trajectory.

4. The system of claim 1, wherein said active capsule endoscope further comprises one or more of an illumination unit or a data transmission unit.

5. The system of claim 1, wherein said Hall-effect sensor array comprises one or more three-axis hall sensors distributed in three-dimensional space of said workspace according to said desired magnetic field.

6. The system of claim 5, wherein each of said one or more three-axis hall sensors are oriented differently with respect to orthogonal coordinates (X, Y, Z) of said desired magnetic field.

7. The system of claim 1, wherein said controller unit comprises an algorithm for calculating said 6D-pose of said active capsule endoscope, χ; said algorithm comprises the steps of:
a. Measuring magnetic field B_{cap} generated by said permanent magnet using said Hall-effect sensor array and representing as a stacked matrix form B_{H}:
$${B}_{H} {=}^{Δ} [ \begin{matrix}{B}_{H}^{11} & … & {B}_{H}^{1 j} \\ ⋮ & ⋱ & ⋮ \\ {B}_{H}^{i 1} & … & {B}_{H}^{i j}\end{matrix} ] i , j ∈ {ℕ}_{+}$$
b. Modelling magnetic field in each Hall-effect sensor in said Hall-effect sensor array as a stacked matrix form B_{c}(χ, Pᵢⱼ) which relative to said 6D-pose, χ, and said position of ij Hall-effect sensor Pᵢⱼ:
$${B}_{c} ( χ , {P}_{i j} ) {=}^{Δ} [ \begin{matrix}{B}_{c} ( χ , {P}_{11} ) & … & {B}_{c} ( χ , {P}_{1 j} ) \\ ⋮ & ⋱ & ⋮ \\ {B}_{c} ( χ , {P}_{i 1} ) & … & {B}_{c} ( χ , {P}_{ij} )\end{matrix} ]$$
c. Measuring orientation of said active capsule endoscope, φ_{I}, θ_{I}, γ_{I}, with respect to a world frame by a filter algorithm;
d. Calculating said 6-DOF pose, X, by solving an optimization function
$$χ = \begin{matrix}arg min \\ χ\end{matrix} ‖ {B}_{H} - {B}_{c} ( χ , {P}_{i j} ) ‖$$
constructed under the condition where φ≡φ_{I}, θ≡θ_{I}, γ≡γ_{I}.

8. The system of claim 7, wherein said B_{c}(χ, Pᵢⱼ) is selected from a model selected from the group consisting of magnetic dipole model, magnetic multipole model, and fitting model of magnetic field in mathematical paradigm.

9. The system of claim 7, wherein said filter algorithm of step (c) is selected from the group consisting of Kalman filter, extended Kalman filter, and complementary filter.

10. The system of claim 1, wherein each of said plurality of magnetic generators is controlled by an input current to generate said desired magnetic field, wherein, at a given position, p, in said desired magnetic field, B, a magnetic force, F, is exerted on said active capsule endoscope based on said input current, C, according to the formula:
$$[ \begin{matrix}B \\ F\end{matrix} ] = [ \begin{matrix}𝔹 ( p ) \\ {m}^{T} {ℬ}_{x} ( p ) \\ {m}^{T} {ℬ}_{y} ( p ) \\ {m}^{T} {ℬ}_{z} ( p )\end{matrix} ] [ \begin{matrix}{c}_{1} \\ ⋮ \\ {c}_{n}\end{matrix} ] = {𝒜}_{B , F} ( m , p ) c ,$$
wherein m is the magnetic moment of said permanent magnet.

11. The system of claim 1, wherein said closed-loop position controller or said vision-based servo controller is a controller selected from the group consisting of a proportional-integral-derivative controller, fuzzy controller, adaptive controller, sliding mode controller, and model predictive controller.

12. The system of claim 1, said control input is formulated based on kinematic analysis of said active capsule endoscope using:
$$R [ \begin{matrix}\dot{{s}_{x}} \\ \dot{{s}_{y}}\end{matrix} ] = {Q}_{s y s t e m} [ \begin{matrix}\dot{ϑ} \\ \dot{φ}\end{matrix} ] with {Q}_{s y s t e m} = {Q}_{image} [ \begin{matrix}{R}_{C}^{{W}^{T}} & O \\ O & {R}_{C}^{{W}^{T}}\end{matrix} ] {Q}_{capsule} ,$$
wherein Q_{capsule} is Jacobian matrix of the capsule; Q_{system} is Jacobian matrix of said system; ν and φ are the heading angles of said active capsule endoscope on the horizontal and vertical plane correspondingly; R is a 2×2 rotation matrix related to the rotational angle around said active capsule endoscope's heading;
$${R}_{C}^{W}$$
is a 3×3 rotation matrix from a world coordinate frame to a camera frame.

13. The system of claim 1, wherein said stomach landmarks comprise one or more of cardia, gastric fundus, gastric body, gastric antrum, gastric angle, and pylorus.

14. The system of claim 1, wherein said pre-planned trajectory comprises a sequence from cardia, gastric fundus, gastric body, gastric antrum, gastric angle, to pylorus.

15. A method for imaging one or more specific structures in a stomach using said system of claim 1, comprising the steps of:
a. Introducing said active capsule endoscope into said stomach;
b. Sending instructions from said controller unit to said actuation unit to generate said desired magnetic field to move said active capsule endoscope according to said pre-planned trajectory; and
c. Capturing one or more images with said active capsule endoscope while moving along said pre-planned trajectory within said stomach.

16. The method of claim 15, wherein said instructions of step (b) are generated by a method comprising the steps of:
i. Identifying a first location of said active capsule endoscope by analyzing one or more images captured by said active capsule endoscope using said image module;
ii. Mapping said one or more images to stomach landmarks;
iii. Obtaining current 6D-pose of said active capsule endoscope;
iv. Calculating discrepancy between said current 6D-pose and said pre-planned trajectory; and
v. Generating instructions for a desired magnetic field to move said active capsule endoscope towards said pre-planned trajectory.

17. The method of claim 15, wherein said stomach is fluid-filled.

18. The method of claim 15, wherein said pre-planned trajectory follows a sequence from cardia, gastric fundus, gastric body, gastric antrum, gastric angle, to pylorus.

19. The method of claim 15, wherein said method further comprises eliminating said active capsule endoscope from said subject using natural peristalsis of the gastrointestinal tract after step (c).

20. The method of claim 15, wherein said method further comprises controlling said active capsule endoscope manually to inspect a region of interest after step (c).
